(19) United States
(12) Patent Application Publication
(10) Pub. No.: US 2012/0031411 A1
(43) Pub. Date: Feb. 9, 2012

(54) ORAL APPLIANCE

(76) Inventor: Richard HORIAN, Corona, CA (US)

(21) Appl. No.: 12/849,955

(22) Filed: Aug. 4, 2010

(51) Int. Cl. A61C 5/14; A61F 5/56

(52) U.S. Cl. 128/848; 128/861; 128/862

(57) ABSTRACT

An oral appliance intended to treat bruxism, snoring, apnea, TMJ and/or palatal lift with a retention line member that uses the interproximal embrasure space between adjacent teeth of the patient user to secure the appliance within the oral cavity as the patient user sleeps. The shape and position of the inter-proximal embrasure space prevents an attached retention line member from dislodging while the user is asleep but permits removal of the appliance upon waking.

### FIELD OF INVENTION

The invention relates to an oral appliance that can be useful for treating the effects of sleep disorders.

### BACKGROUND OF THE INVENTION

Sleep disorders adversely affect the sleep of many. A person suffering from such as bruxism, or “teeth-grinding,” generally clenches and grinds his/her teeth while sleeping. The effects of such nocturnal activity may manifest a variety of unpleasant effects that include headaches, stiff neck, receding gums, misshapen and misaligned teeth, worn teeth, and/or sleeplessness. The typical treatment for bruxism is a mouthpiece, like an athletic mouthpiece that has been formed for the user by softening the appliance in boiling water and which is worn over the top and/or bottom teeth while sleeping. Forces generated due to the clenching and grinding of the teeth are then distributed to, and mitigated by, the mouthpiece.

Those suffering from sleep apnea, a potentially life-threatening disruption of breathing while asleep, may be unaware that they suffer episodes of choking or gasping for breath hundreds of times each night. Symptoms include frequent silences in sleep due to cessation of breathing, choking or gasping during sleep, loud snoring, sudden awakenings, waking up in a sweat, and daytime sleepiness due to the lack of restful sleep at night. Some forms of sleep apnea due to obstruction can be treated with an oral appliance.

Snoring devices are removable dental appliances that are worn in the mouth while sleeping. They are intended to reduce airway obstruction and improve airflow. Exemplary categories of such devices include palatal lifts, tongue-retaining devices and mandibular repositioning appliances.

The purpose of oral appliances intended for treatment of sleep disorders is to shape, reposition or align one or more of the physical anatomical structures that are located within the human oral cavity to correct, mitigate or rearrange structures so as to treat the symptoms and/or causes of the disorder. There have been literally hundreds of mechanical appliances to treat bruxism, sleep apnea, and snoring. Examples include those devices shown and described in U.S. Pat. Nos. 4,169,473; 5,467,783 and 5,692,521, the disclosures of which are herein incorporated by reference. The prior appliances have been made available in both stock, pre-configured models as well as custom models that are designed specifically to fit within the oral cavity of a specific patient.

Prior art oral appliances typically share the common problems of: (a) an uncomfortable size that is sufficient to affect restful sleep of the patient, and (b) inadvertent ejection during sleep from natural movements of the sleeping patent's mouth or, more commonly, subconscious use of the tongue to dislodge and then expel the appliance.

A comfortable, effective oral appliance that stays comfortably in place through restful sleep is not an easy task. There are many reasons for this:
First, no two oral cavities are the same. Differences between individuals can be quite extensive for any conformed shaped appliance to apply.
Second, the lateral and bilateral movement of the lower jaw against the fixed position of the upper jaw make it difficult to position an appliance and keep it in proper position.
Third, the bite force of the jaw is often sufficient to rule out many designs and materials that would provide good comfort but lack sufficient durability. An oral appliance that could find itself between moving jaws must be able to withstand hundreds of pounds of pressure.
Lastly, the appliance must be able to resist the effects of the human tongue. The human tongue is a miracle of mechanics; it is able to move or conform itself into a seemingly infinite combination of shapes that can apply pressure to dispel or dislodge almost any oral obstruction.

### SUMMARY OF THE INVENTION

It is an objective of the invention to provide an oral appliance that is effective for the treatment of symptoms of common sleep disorders but that allows comfort while wearing the appliance during sleep so that the required rejuvenating rest during sleep is not materially disturbed.

It is an additional objective of the invention to provide a method to hold or anchor an oral appliance within the oral cavity so that it cannot be dispelled during sleep.

It is a further objective of the invention to create an oral appliance that is extremely easy and fast to install and or remove to promote use when one is most ill equipped to perform a complex mechanical procedure; just before and after sleep.

In accordance with these and other objectives of the invention that will become apparent from the description herein, an oral appliance according to the invention is provided that will shape, reposition and/or align anatomical structures within an oral cavity of a human patient and remain in position while the patient sleeps, said oral appliance comprising a retention member secured at one end to said oral appliance and having a second end that extends into or through an interproximal embrasure space between adjacent teeth of said patient and anchors said appliance during sleep.

An especially preferred oral appliance comprises: (a.) a durable spacing member having a generally planar shape of an extended length with a proximal end that is shaped to be insertable into the oral cavity of the patient and a distal end; and (b.) a retention line member that is secured at said proximal end and which exhibits a diameter that is sufficiently small to pass at least into and, preferably, through an inter-proximal embrasure space between adjacent teeth of said patient and with a length sufficient to be useful to secure said spacing member within said oral cavity.

The oral appliance of the present invention resolves problems afflicting prior oral appliances and mouth guards. By using an inter-proximal embrasure space between adjacent teeth, the appliance can be positioned and held in place with sufficient reliability to resist dislocation during sleep despite the actions of mandible and tongue. Such a positioning system permits the user to install and remove the appliance with little need for extraordinary dexterity or a mirror.

### BRIEF DESCRIPTION OF THE DRAWINGS

FIG. 1 is a view of an appliance according to the invention with a rigid or semi-rigid retention member that extends from one end of the appliance.

FIG. 2 shows an alternate design for an appliance according to the invention with a rigid or semi-rigid retention member having a hook-shaped proximal end.

FIG. 3 illustrates a further alternate embodiment of an appliance according to the invention in which the appliance also includes a securing member in the form of a hinged snap-closure arm that will secure the retention line member in position beneath the snap-closure arm.

FIG. 4 shows an embodiment of an appliance according to the invention having a securing member in the form of a threaded thumbscrew that is advanced into a hole in the spacing member until it secures the retention line member in position.

FIG. 5 depicts a further view of an embodiment in which the threaded portion of the threaded thumbscrew securing member extends into a relatively thick portion of the appliance to protect the threads from contact with oral tissues and surfaces.

FIGS. 6 and 7 are top and side views, respectively, of an appliance according to the invention having an integral living hinge with secured positioned magnets that will secure the retention line between them when hinge is folded to allow magnets to pinch secure the retention line between them.

FIGS. 8-12 are views of a user's oral cavity and how the appliance of the present invention is secured therein for treatment of symptoms associated with sleep disorders such as bruxism, TMJ, sleep apnea and snoring during sleep. FIG. 8 shows the appliance of FIG. 1 in use to affect palatal lift useful in the treatment of snoring as well as speech disorders.

FIG. 9 shows how the appliance of FIGS. 5 and 6 is inserted into the oral cavity to allow the retention line to be positioned behind the teeth so the retention line may be pulled forward to pass through the inter-proximal space embrasure.

FIG. 10 shows how the retention line is secured once through the inter-proximal space embrasure. The living hinge secured magnets are folded pinching and securing the retention line between the magnets.

FIG. 11 shows the appliance of FIGS. 4 and 5 in fully secured position use. The retention line is passed through the inter-proximal space embrasure from behind the teeth and pulled forward. The retention line then passes under the head of the threaded thumbscrew. The thumbscrew is then tightened to secure the retention line to hold the appliance firmly in place between the upper and lower jaw. This is a preferred treatment for bruxism. Teeth grinding is prevented with the appliance preventing the teeth of the upper jaw coming into contact with the lower jaw.

FIG. 12 shows the appliance of FIG. 2 in use. The appliance is placed diagonally between the back underside of the tongue and the upper teeth. The rigid or semi-rigid retention hook is passed through the inter-proximal space embrasure of the upper teeth. Each time the jaw is attempted to close the lever acts to lift the entire lower palate. The hook through the upper teeth embrasure secures the device. This is useful in treating more advanced cases of apnea and or snoring.

### DETAILED DESCRIPTION OF THE INVENTION

Oral appliances according to the present invention can be of virtually any shape provided that the appliance includes a retention line member that is secured at a first end to said appliance and includes a second end that extends into or though through an interproximal embrasure space between adjacent teeth of the user patient. Embrasure spaces between either upper or lower teeth can be used, depending on the shape and desired position of the appliance while the patient user sleeps.

Retention line members of the invention can be formed into the body materials of the oral appliance, secured therein after construction or inserted into the appliance after installation by the patient user. Such flexibility permits the conventional “boil-and-bite” mouthpiece appliances to be modified to include a retention line member. Examples of such embodiments include: (a) molding a retention line member into the mouthpiece at the time of manufacture; (b) providing an opening in the mouthpiece through which a separate retaining line member can be inserted by the patient user and secured in place to retain the mouthpiece during sleep, or by (c) providing the mouthpiece of a first thermoplastic material in one or more areas of the mouthpiece that will form the retention line member in situ.

In situ formation of the retention line member in a “boil-and-bite” mouthpiece with an obverse (front), reverse (rear) and bite surface disposes a portion of a second thermoplastic material on the obverse side of the mouthpiece that is adjacent the front portions of the user's teeth. The second thermoplastic material preferably exhibits a softening point temperature that is different, e.g., more or less, than that of the first thermoplastic material from which the body of the mouthpiece is made so that, when heated to temperatures of less than 100° C. and preferably within the range of 70°-100° C., the second thermoplastic will deform sufficiently to exude into at least one embrasure space between one or more pairs of adjacent teeth of the patient user and form a retaining line member in situ of an abbreviated length as the mouthpiece is formed to the user's teeth. When the mouthpiece is cooled, the retention line member and mouthpiece become fixed in shape. This retaining line member will resist subconscious removal as the patient user sleeps yet allow conscious removal upon wakening and a comfortable fit.

A preferred appliance according to the invention is made with a durable, generally planar, generally rectangular, spacing member that has a proximal end which is inserted into a user's mouth and is intended to remain in position while asleep. The distal end of the spacing member extends to, or past, the user's teeth depending on the nature of the disorder that is treated with the appliance. A retention line member is attached to the proximal end of the appliance and has a length and diameter sufficient to extend toward the mouth and through the inter-proximal space between at least two adjacent teeth of the upper or lower jaw. The retention line member is then secured to the teeth of the upper or lower jaw or to the spacing member by a securing member on the spacing member.

The spacing member that forms the contact surface in the preferred oral appliance of the invention may be made of any material, composite or combination of materials that is sufficiently tough to withstand degradation from repeated contact with crushing and/or moving teeth while presenting sufficiently soft contact surfaces that dental and oral surfaces are not harmed or abraded as a result of such contact. Suitable materials include natural and synthetic polymers and copolymers.

Preferred materials for the spacing member and/or its contact surfaces include nylon, polypropylene, polyethylene, polytetrafluoroethylene, copolymers and silicone rubbers. These materials are well known for their slippery surface finish and they are not as rigid or brittle as many other plastics. Thus the teeth, when they grind or bear down are doing so against an extremely durable, resilient and slippery surface to allow easy transverse movement of the teeth with minimal friction.

The inter-proximal space in the human mouth is the triangular area at the top of and between the teeth. Part of the inter-proximal space is occupied by the inter-dental papilla. The inter-dental papilla is a triangular fold of gingival tissue. The part of the inter-proximal space not occupied is called the embrasure. The embrasure occupies an area bordered by interdental papilla, the proximal surfaces of the two adjacent teeth, and the contact point. If there is no contact point between the teeth, then the area between them is called a diastema instead of an embrasure. However, the teeth in most people include an embrasure and even those with diastema include a gap that is more narrow than the embrasure space at the top of adjoining teeth.

The patient user of an oral appliance according to the invention has many choices of front or lower teeth inter-proximal embrasure spaces to choose for the best and most comfortable fit of the retention line member. The front upper and front lower teeth consist of two central incisors, two lateral incisors and two cuspids to easily position the wire through the most advantageous inter-proximal embrasure space. Therefore, there are a total of 10 front inter-proximal embrasure spaces where the positioning wire may be easily and conveniently located.

By using the inter-proximal embrasure space to both position as well as secure a mouth guard, it was possible to achieve function, comfort and ease of use for a mouthguard. A considerable amount of research was conducted over several years to locate a single point within the oral cavity universal to most people that could be used to position and hold the appliance or mouth guard. Clamps, springs, wedges and formed shapes where considered in a variety of locations within the mouth including gums, palate, roof, lip interior and fixing to the exterior shape of each tooth or groups of teeth. None of them worked well until the present invention was developed.

The retention line member can be made of virtually any material that will enable the retention line member to pass through a user's embrasure between adjacent teeth of the upper jaw and allow the appliance to be secured in place. The inter-proximal embrasure space that exists between the teeth of most people is 0.065 inches (16.5 mm) or less. Suitable forms for the retention line member include rigid, semi-rigid or flexible filaments, yarns, monofilaments, braids, tapes or ribbons. Suitable materials for the retention line member include metals, polymers, polymer coated metals, and composites that are resistant to the various fluids, enzymes and tissues in the human mouth while avoiding abrasion and/or damages to the tissues and dental structures found therein.

It is advantageous to make the retention line member as thin as possible while still providing adequate tenacity and ease of use. If the retention line member is in the form of a filament, yarn, or monofilament, the retention line member preferably exhibits an outer diameter within the range of about 0.005 inches to about 0.2 inches (0.1-5 mm) and even more preferably an outer diameter within the range of about 0.01 inches to about 0.05 inches (0.25-1.3 mm). Tapes or ribbon retention line members should have a thickness of less than about 0.065 inches (1.6 mm) and may have a width within the range from about 0.010 inches to about 0.25 inches (0.4-6 mm). Research indicated that a wire diameter of 0.010″-0.015″ (0.25-0.4 mm) was an especially preferred size range that allowed the best strength, flexibility, maneuverability, wearing comfort and ease of handling to secure the retention line member.

A preferred embodiment for a retention line member for the present invention is a 0.012 inch (0.30 mm) diameter monofilament made of fluorcarbon. This material provides the resilience, strength and flexibility required for long use and easy insertion through the embrasure space.

Regardless of closure method, the retention line member that passes through the inter-proximal embrasure space performs a critical function. Primarily, it secures the oral appliance within the oral cavity. Secondarily, the retention line member allows some relative front-to-back and side-to-side movement by the restrained spacing member so a feeling of claustrophobic enclosure or pressure within the oral cavity is not experienced. This can provide the user with a relatively comfortable experience with the appliance while asleep. In fact, one can get used to wearing the appliance of the present invention very quickly without sleep obstruction.

The invention is conveniently described in the context of the drawing figures attached hereto. Similar structures are identified with the same reference number.

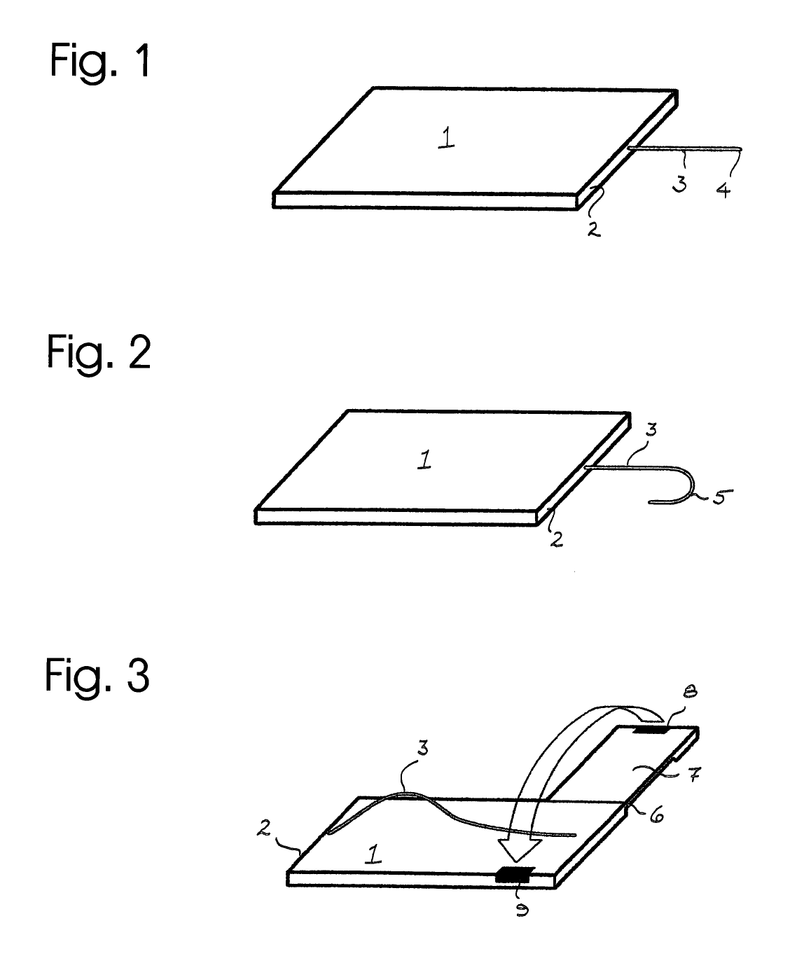

FIG. 1 shows a spacing member 1 as a generally planar, rectangular plate of approximately 0.850 inches (21 mm) wide by about 2.25 inches (56 mm) long by about 0.125 inches (3 mm) thick. The specific shape of spacing member 1 is not, however, critical to the present invention and can exhibit virtually any shape that will fit into the user's oral cavity and achieve a position to help treat a condition suffered by the user.

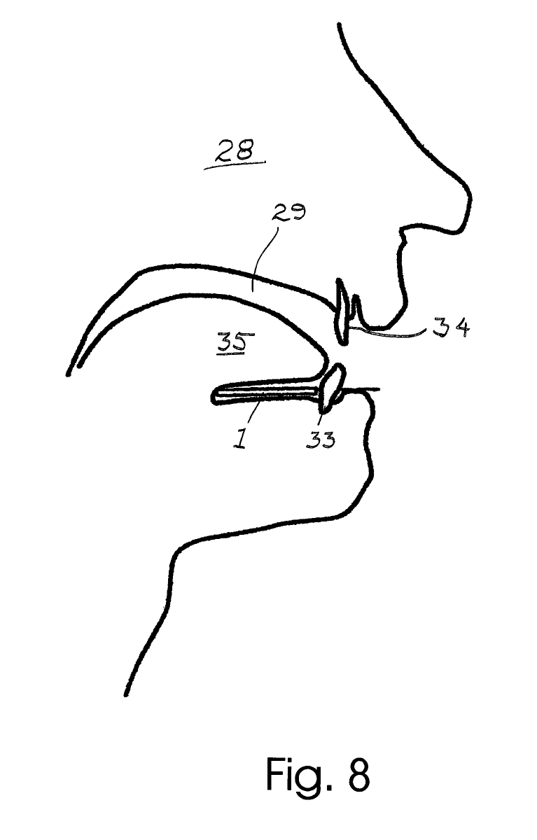

Proximal end 2 is intended to be positioned within the mouth of a user (see FIGS. 8-12) with a flexible, rigid or semi-rigid retention line member 3 protruding from proximal end 2. As shown, retention line member 3 is a flexible, rigid, semi-rigid filament, yarn, monofilament or braided yarn of a durable material that is safe to use in oral cavities and that will not harm dental surfaces or tissues within the oral cavity but which exhibits adequate tenacity to secure the appliance within a user's mouth while asleep. Retention line member 3 does not exhibit any beads, shapes, formed bends or devices at its distal end 4.

As shown in FIG. 2, retention line member 3 may be formed to include a hook-shaped terminal end 5 that can pass through the embrasure space and hook over the surface of a tooth to remain secure and in place within the user's mouth while asleep.

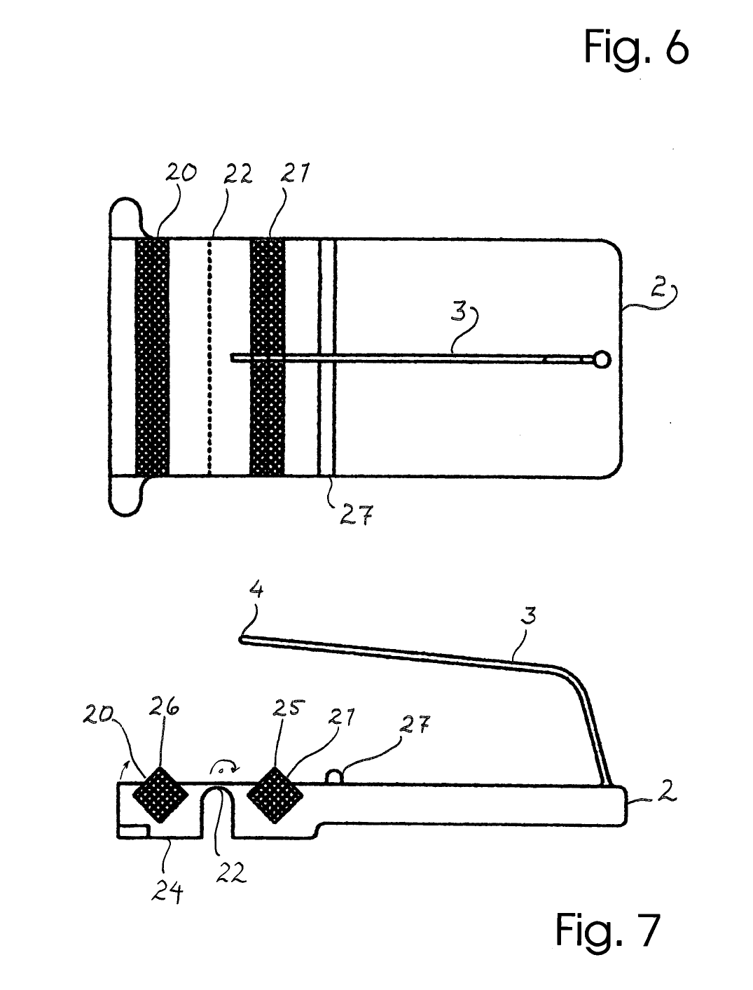

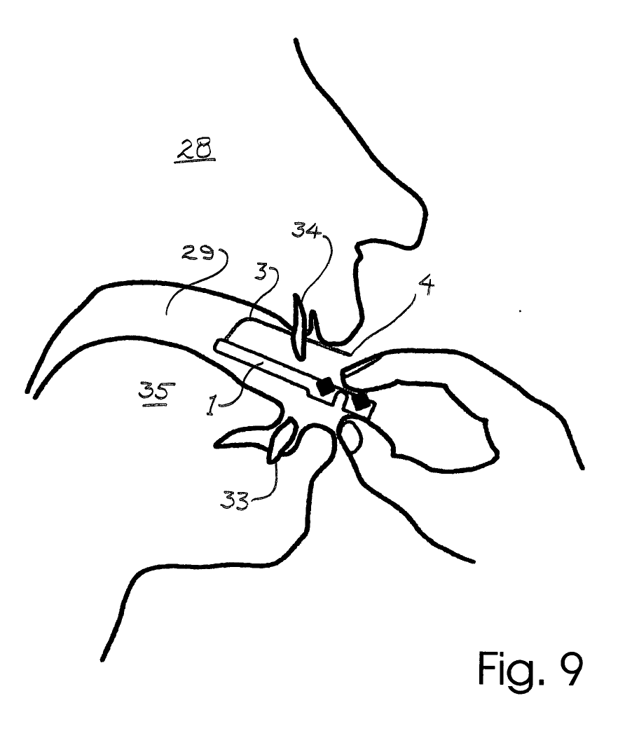

FIG. 3 illustrates spacing member 1 that has been formed to include living hinge 6 and snap-close arm 7 with overhanging snap closure 8 that mating engages in a releasable connection with undercut recess 9. If desired, closure 8 and recess 9 may be replaced with embedded magnets of opposite facing polarity so that the attraction of the opposing poles will hold arm 7 in position. When retention line member has been passed through an embrasure between adjacent teeth in the upper jaw of a user (see, FIGS. 9-12), retention line member 3 can be secured in position by the compressive force between arm 7 and spacing member 1 as held by the mating interaction of snap closure 8 and undercut recess 9. Alternatively, snap closure 8 and undercut recess 9 can be replaced by suitably embedded magnets of opposing polarity (not shown) that will attract and remain in a closed position with retention line member 3 secured therebetween. The use of a magnet closure to secure retention line member 3 is also shown in FIGS. 6 and 7.

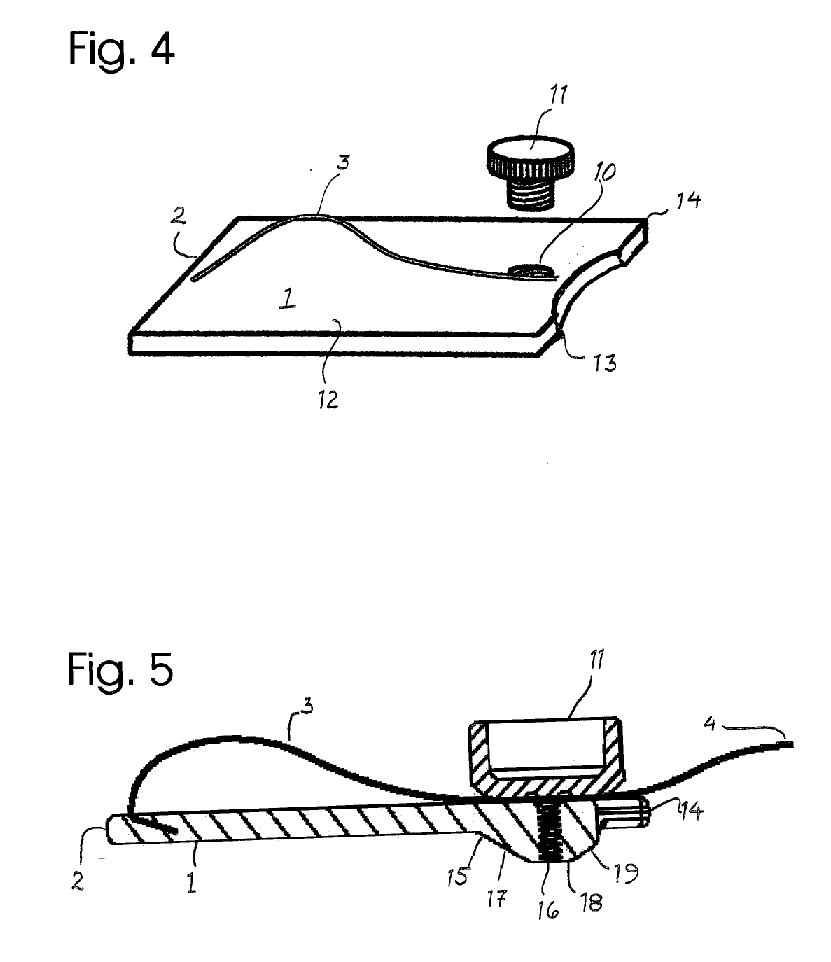

FIG. 4 depicts a transverse hole 10 through spacing member 1. Hole 10 is dimensioned to permit threaded thumbscrew 11 to advance into hole 10 and secure retention line member 3 between thumbscrew 11 and upper surface 12 of spacing member 1. To remove the device, the user would loosen thumbscrew 11 to free retention line member 3 from under the head of thumbscrew 11 and gently retract retention line member 3 through the inter-proximal embrasure space to remove the entire device from the oral cavity. If desired, curved concave portion 13 can be formed into spacing member 1 at distal end as a finger grip.

If thumbscrew 11 is used as a securing member, it is preferable that spacing member 1 include a forward portion 15 near distal end 14 that is sufficiently thick to surround and enclose threads 16 of thumbscrew 11 that might otherwise extend through spacing member 1. As shown, forward portion 15 includes thickening gradient portion 17, central portion 18 and tapering gradient portion 19.

FIGS. 6 and 7 show top and side views of an embodiment that uses a pair of distal and proximal magnets 20, 21 on either side of a living hinge 22 formed into the body of spacing member 1 to secure retention line member 3. The proximal and distal sides 23, 24 of spacing member 1 each include a powerful, rectangular-shaped, magnet that are installed into spacing member 1 on edge to present an edge 25, 26 of each that projects away from the upper surface of spacing member 1. Magnets 20, 21 are held in place by corresponding diamond shape channels that have been molded into proximal and distal ends 23, 24 of spacing member 1.

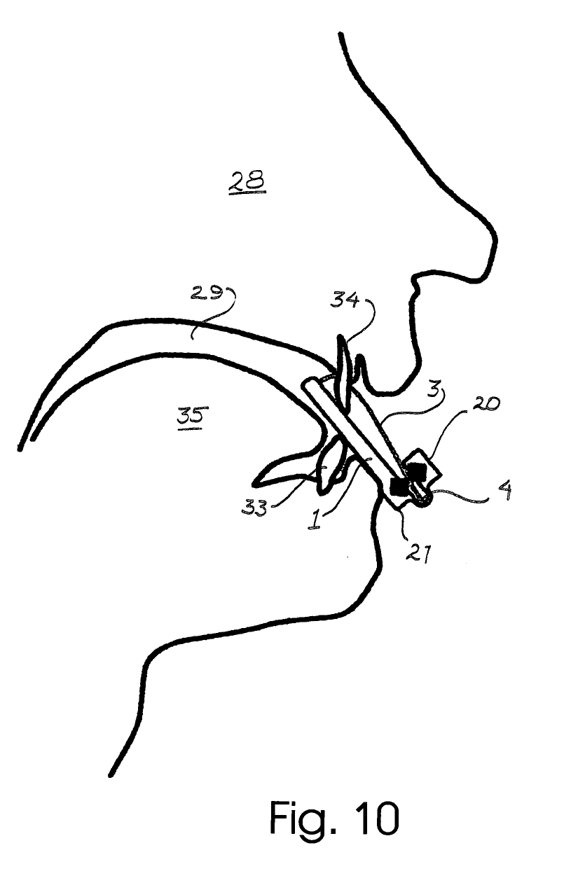

Living hinge 22 is formed into spacing member 1 as a portion of relatively thinner material whereby rotation of distal end 24 around living hinge axis 26 allows distal magnet 20 to approach proximal magnet 21. When the poles of magnets 20, 21 are properly aligned, the magnets will attract and can be used to secure retention line member 3 therebetween. The user simply passes retention line member 3 through his/her inter-proximal embrasure space and places the distal end of retention line member 3 anywhere on top of proximal magnet 21. A flip of the finger rotates distal end 24 around living hinge 22 and into a position or distal to proximal magnet 21 to secure retention line member 3 between magnets 20, 21. See, FIGS. 9 and 10.

If desired, proximal ridge 27 can be formed into spacing member 1 as an additional abutment surface that would cause retention line member 3 to bend some additional amount in order to increase the effectiveness of the retention forces of magnets 20, 21.

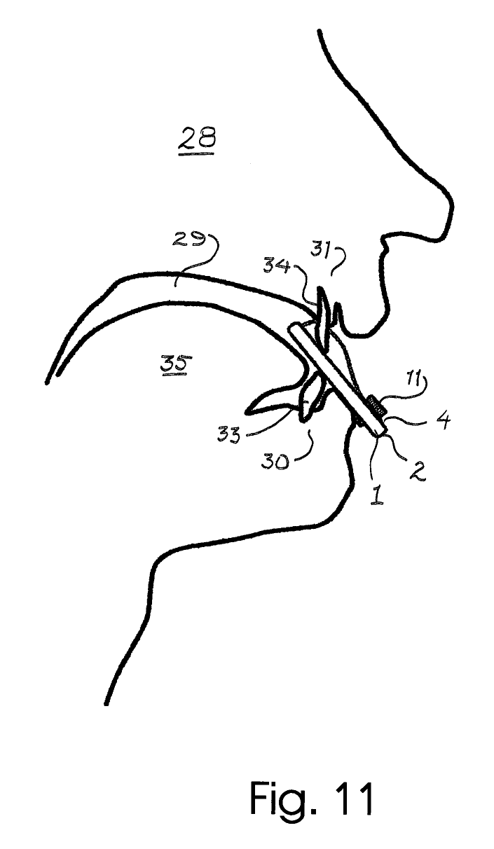

FIGS. 9-11 illustrate the use of the present oral appliance for treating bruxism. As shown, distal end 4 of retention line member 3 is passed by the user 28 through an inter-proximal embrasure space in his/her upper jaw and then secured to spacing member 1, such as with attracting magnets on a living hinge (FIGS. 9, 10) or a threaded thumbscrew (FIG. 11). Proximal end 2 is then positioned within oral cavity 29 at the back side of the user's upper teeth. When user 28 bites down or grinds the lower jaw 30 against the upper jaw 31, spacing member 1 is positioned and held between the teeth to mitigate the adverse effects of such actions.

In the event the user has a more convenient embrasure between adjacent teeth of the lower jaw 30, it should be understood that retention line member 3 may be inserted there so that the position of spacing member 1 is flipped top-to-bottom and used in an orientation that would be considered upside down from the orientation described in the present figures.

Further, the present appliance need not be installed or entirely contained within the oral cavity. The proximal end of the appliance can be located behind the user's teeth while the greater length of the appliance is outside the oral cavity. The majority of the treating member passes over the bottom teeth (or upper teeth if reversed) and lies outside the mouth to provide the most comfortable nights sleep possible wearing such an oral appliance intended for the treatment of bruxism and TMJ.

FIG. 8 shows how spacing member 1 from FIG. 1 is used to affect palatal lift useful in the treatment of snoring as well as speech disorders. As shown, spacing member 1 is positioned behind lower teeth 33 and at the back underside of the user's tongue 35. Retention line member 3 is passed through an embrasure space between adjacent teeth in lower jaw 30. Please note that retention line member 3 need not be secured to a securing member associated with spacing member 1 of an external structure, cleat, or by a separate device as passage of retention line member 3 through an embrasure space. It is often adequate if spacing member 1 abuts the user's teeth in the front and abuts against tongue 35 to keep spacing member 3 in place while the user sleeps.

FIG. 9 shows how spacing member 1 from FIGS. 6 and 7 is inserted into oral cavity 29 and then positioned behind the teeth 33, 34 of jaws 30, 31 so the retention line member 3 may be pulled forward to pass through an inter-proximal space embrasure.

FIG. 10 shows how the retention line is secured once through the inter-proximal space embrasure. The living hinge is secured in position by magnets that are folded to pinch and securing the retention line between the magnets.

FIG. 11 shows the appliance of FIGS. 4 and 5 in fully secured position use. The retention line is passed through the inter-proximal space embrasure from behind the teeth and pulled forward. The retention line then passes under the head of the threaded thumbscrew. The thumbscrew is then tightened to secure the retention line to hold the appliance firmly in place between the upper and lower jaw. This is a preferred treatment for bruxism. Teeth grinding is prevented with the appliance preventing the teeth of the upper jaw coming into contact with the lower jaw.

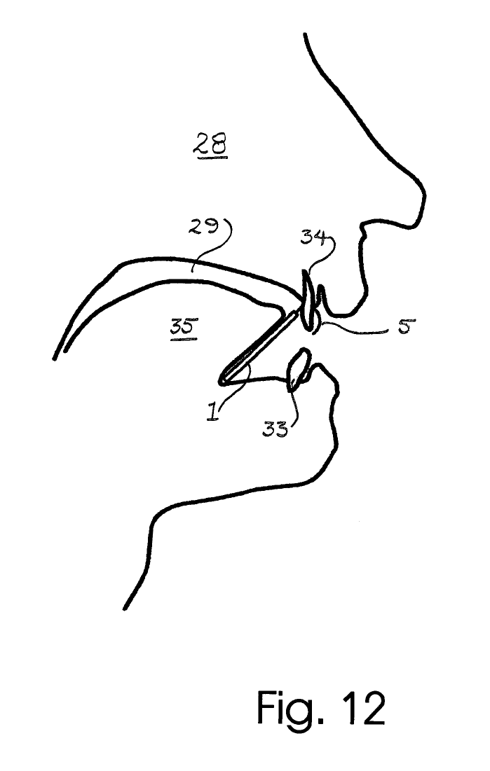

FIG. 12 shows the appliance of FIG. 2 in use. The appliance is placed diagonally between the back underside of the tongue and the upper teeth. The rigid or semi-rigid retention hook is passed through the inter-proximal space embrasure of the upper teeth. Each time the jaw is attempted to close the lever acts to lift the entire lower palate. The hook through the upper teeth embrasure secures the device. This is useful in treating more advanced cases of apnea and or snoring.

The appliance of the invention can be used to treat snoring and sleep apnea. Using the appliance of FIG. 2 with a hook-shaped end 5 and as shown in FIG. 12, spacing member 1 is inserted into oral cavity 9 so that proximal end 2 is wedged under tongue 32 and the hook-shaped end 5 passes through an embrasure of the upper teeth and around one of the adjacent teeth. Once installed, spacing member 1 cannot be dislodged by action of tongue 35 or relative motion of the user's upper and lower jaws. The contact point between the two teeth in which the wire is positioned prevents the wire from passing through to allow release of the member. Spacing member 1 must be manually dislodged to be removed. The diagonal position of spacing member 1 gently lifts the tongue and opens the user's airway.

Once those skilled in the art are taught the invention, many variations and modifications are possible without departing from the inventive concepts disclosed herein. The invention, therefore is not to be restricted except in the spirit of the appended claims.

What is claimed:

1. An oral appliance that will shape, reposition and/or align anatomical structures within an oral cavity of a human patient suffering from a mandibular and/or sleep disorder, said appliance comprising a retention line member securedly attached to said appliance at a first end and exhibiting dimensions sufficient to secure said appliance by interaction with an interproximal embrasure space between adjacent teeth of patient user while said patient user is asleep.

2. An oral appliance according to claim 1, wherein said retention line member is sufficiently small to pass into or through said inter-proximal embrasure space.

3. An oral appliance according to claim 1, further comprising a securing member that can secure said retention line member.

4. An oral appliance according to claim 1 wherein said retention line member has been formed in situ from a second thermoplastic material disposed on an obverse side of said appliance that is adjacent an interproximal embrasure space of said patient user.

5. An oral appliance according to claim 4 wherein said second thermoplastic material exhibits a softening point different than said first thermoplastic material.

6. An oral appliance according to claim 1 wherein said appliance is shaped as a generally rectangular, generally planar, spacing member.

7. An oral appliance according to claim 6 wherein said spacing member further comprises a closure member attached by a living hinge to a lateral side of said spacing member.

8. An oral appliance according to claim 7 further comprising a threaded thumbscrew and wherein said spacing member exhibits an opening therein that is generally transverse to said planar shape whereby said threaded thumbscrew can be advanced into said opening in order to secure said retaining line member in a secured position.

9. A method for treating a sleep condition malady in a human patient user with steps that comprises: (a) inserting an oral appliance into an oral cavity of said patient user; and (b) securing said appliance therein with a retention line member extending between said oral appliance and at least one interproximal embrasure space between adjacent teeth of said user patient.

10. A method according to claim 9 wherein said oral appliance is a mouthpiece that covers obverse, reverse and cutting surfaces of teeth in a jaw of said patient user.

11. A method according to claim 9 wherein said mouthpiece is made with a first thermoplastic that softens when immersed in boiling water and can then mold to teeth in said jaw.

12. A method according to claim 11 wherein said retention line member is formed in situ from a second thermoplastic material disposed on said mouthpiece.

13. A method according to claim 9 wherein said oral appliance comprises: (a) a durable spacing member having a generally planar shape of an extended length with a proximal end that is shaped to be insertable into the oral cavity of the patient; and (b) a retention line member secured at said proximal end, said retention line member exhibiting a diameter that is sufficiently small to pass into or through said inter-proximal embrasure space.

14. A method according to claim 9 wherein said oral appliance is inserted into the oral cavity so that its proximal end is wedged under the tongue of the patient user to lift the tongue and opens user airways.

15. A method according to claim 9 wherein said oral appliance is inserted into the oral cavity so that, when the user patient bites down or grinds the lower jaw against the upper jaw, the spacing member is positioned between the teeth to mitigate the adverse effects of such actions.
